(19) United States
(12) Patent Application Publication
(10) Pub. No.: US 2007/0043005 A1
(43) Pub. Date: Feb. 22, 2007

(54) TREATMENT OF HYPERPROLIFERATIVE DISEASES USING HIGH DOSES OF ACTIVE VITAMIN D

(75) Inventor: Richard B. Mazess, Montecito, CA (US)

Correspondence Address: MICHAEL BEST & FRIEDRICH, LLP, ONE SOUTH PINCKNEY STREET, P O BOX 1806, MADISON, WI, 53701, US

(73) Assignee: Genzyme Corporation, Cambridge, MA (US)

(21) Appl. No.: 11/382,887

(22) Filed: May 11, 2006

Related U.S. Application Data
(63) Continuation of application No. 09/995,911, filed on Nov. 28, 2001, now abandoned.
Application No. 09/995,911 is a continuation-in-part of application No. 09/891,814, filed on Jun. 26, 2001, now Pat. No. 6,503,893.
Application No. 09/891,814 is a continuation-in-part of application No. 09/596,149, filed on Feb. 23, 1998, now Pat. No. 6,537,982.
Application No. 09/596,149 is a division of application No. 08/781,910, filed on Dec. 30, 1996, now Pat. No. 5,763,429.

(51) Int. Cl. A61K 31/59

(52) U.S. Cl. 514/167

(57) ABSTRACT

The present invention provides a method of inhibiting the hyperproliferative cellular activity of neoplasms and other hyperproliferative diseases with an active vitamin D compound utilizing a high dose, episodic treatment protocol.

### CROSS-REFERENCE TO RELATED APPLICATIONS

This application is a continuation of U.S. patent application Ser. No. 09/995,911 filed Nov. 28, 2001, which is a continuation-in-part of U.S. patent application Ser. No. 09/891,814 filed Jun. 26, 2001, now U.S. Pat. No. 6,503,893, which is a continuation-in-part of U.S. patent application Ser. No. 09/596,149, filed Feb. 23, 1998, now U.S. Pat. No. 6,537,982, which is a divisional of U.S. patent application Ser. No. 08/781,910, filed Dec. 30, 1996, now U.S. Pat. No. 5,763,429, all of which are incorporated herein by reference.

### STATEMENT REGARDING FEDERALLY SPONSORED RESEARCH OR DEVELOPMENT

Not Applicable

### BACKGROUND OF THE INVENTION

This invention relates to a method of treating hyperproliferative diseases utilizing active forms of vitamin D. The active vitamin D compound inhibits the hyperproliferative cellular activity of these diseases and promotes differentiation of the cells with reduced risk of hypercalcemia. The reduced risk of hypercalcemia is achieved 1) by episodic administration of high dose active vitamin D; or 2) by episodic co-administration of the active vitamin D with an antihypercalcemic agent such as a bisphosphonate. The risk is further mitigated where the active vitamin D compound is a hypocalcemic active vitamin D. The present invention also provides a pharmaceutical combination therapy in which the active vitamin D compound is co-administered with other antineooplastic (i.e., anticancer) agents. The methods of present invention are also useful in controlling, stabilizing or reducing serum parathyroid hormone related protein (PTHrP) levels produced by malignant cells, and thus, the hypercalcemia associated therewith.

Extensive research during the past two decades has established important biologic roles for vitamin D apart from its classic role in bone and mineral metabolism. Specific nuclear receptors for 1α,25-dihydroxyvitamin D₃, the hormonally active form of vitamin D, are present in cells from diverse organs not involved in calcium homeostasis. For example, specific, biologically active vitamin D receptors have been demonstrated in the human prostatic carcinoma cell line, LNCaP, (Miller et al., 52 Cancer Res. (1992) 515-520); vitamin D receptors have also been described for many other neoplastic cells, e.g., carcinomas of the breast and the colon.

It has been reported that certain vitamin D compounds and analogues are potent inhibitors of malignant cell proliferation and are inducers/stimulators of cell differentiation. For example, U.S. Pat. No. 4,391,802 issued to Suda et al. discloses that 1α-hydroxyvitamin D compounds, specifically 1α,25-dihydroxyvitamin D₃ and 1α-hydroxyvitamin D₃, possess potent antileukemic activity by virtue of inducing the differentiation of malignant cells (specifically leukemia cells) to nonmalignant macrophages (monocytes), and are useful in the treatment of leukemia. Antiproliferative and differentiating actions of 1α,25-dihydroxyvitamin D₃ and other vitamin D₃ analogues have also been reported with respect to cancer cell lines. More recently, an association between vitamin D receptor gene polymorphism and cancer risk has been reported, suggesting that vitamin D receptors may have a role in the development, and possible treatment, of cancer.

Previous studies of vitamin D compounds and cancer treatment have focused exclusively on vitamin D₃ compounds. Even though these compounds may indeed be highly effective in promoting differentiation in malignant cells in culture, their practical use in differentiation therapy as anticancer agents is severely limited because of their equally high potency as agents affecting calcium metabolism. At the levels required in vivo for effective use as, for example, antileukemic agents, these same compounds can induce markedly elevated and potentially dangerous blood calcium levels by virtue of their inherent calcemic activity. That is, the clinical use of 1α, 25-dihydroxyvitamin D₃ and other vitamin D₃ analogues as anticancer agents is precluded, or severely limited, by the risk of hypercalcemia. This indicates a need for compounds with greater specific activity and selectivity of action, i.e., vitamin D compounds with antiproliferative and differentiating effects but which have less calcemic activity.

In addition to the risk of hypercalcemia associated with clinical use of certain vitamin D compounds that are potent stimulators of intestinal calcium absorption, hypercalcemia has now also been specifically associated with malignancy. Such malignancy associated hypercalcemia (MAH) is often a major contributor to morbidity and complicates clinical management of the malignancy. Parathyroid hormone related protein (PTHrP) is one of the main causative substances of such hypercalcemia, and is overproduced by malignant cells. PTHrP is closely related to parathyroid hormone (PTH) and binds to the same receptor as PTH as well as other receptors. 1,25-dihydroxyvitamin D₃ has been found to repress the transcription of the PTHrP gene in cells; however, the 1,25-dihydroxyvitamin D₃ compounds themselves increase serum calcium levels. Accordingly, a need also exists for specific treatment regimens of active vitamin D that will provide antiproliferative and differentiating effects yet control PTHrP levels.

### SUMMARY OF THE INVENTION

The present invention provides a method of treating hyperproliferative disease conditions, such as those characterized by hyperproliferative cell growth and/or abnormal cell differentiation, with reduced risk of hypercalcemia. The method includes use of active vitamin D compounds (defined hereinafter), and of particular value, hypocalcemic active vitamin D compounds, especially of vitamins D₂ and D₄, in high dosage form, administered on an intermittent or episodic basis, to inhibit abnormal cell growth and promote cell differentiation. The active vitamin D compound may be used as sole therapy or may be used in combination therapy with one or more other antineoplastic agents. An antihypercalcemia agent may also be used with the active vitamin D or with the vitamin D-antineoplastic agent combination. A high dosage episodic regimen of active vitamin D is also of value in controlling serum PTHrP level, the elevation of which is correlated with hypercalcemia associated with malignancies or hyperproliferative diseases.

The foregoing, and other advantages of the present invention, are realized in one aspect thereof in a method of inhibiting the hyperproliferative activity of neoplastic or hyperplastic cells, comprising treating the cells with an effective amount of an active vitamin D compound. The treating step includes inhibiting proliferation of, and inducing and enhancing differentiation in such cells. The effective amount of the active vitamin D is provided by a high dose, episodic administration regimen. The methods of the present invention are also of value in controlling levels of PTHrP and the hypercalcemia associated with malignancies.

The vitamin D compound of the present invention is an active vitamin D and is generally represented by the formula (I) described hereafter. The active vitamin D compounds of the present invention include vitamin D compounds having a hydroxy group substituted in at least one of the C₁, C₂₄ or C₂₅ positions of the molecule, i.e., a hydroxy vitamin D. For example, compounds of formula (I) suitably include, without limitation, α,24-dihydroxyvitamin D₂, 1α,24-dihydroxyvitamin D₄, 1α,25-dihydroxyvitamin D₄, 1α,25-dihydroxyvitamin D₂, 1α,25-dihydroxyvitamin D₃, and 1α,24,25-trihydroxyvitamin D₂, and also include such pro-drugs or pro-hormones as 1α-hydroxyvitamin D₂, 1α-hydroxyvitamin D₄, 1α-hydroxyvitamin D₃, 24-hydroxyvitamin D₂, 24-hydroxyvitamin D₄, 24-hydroxyvitamin D₃, and 25-hydroxyvitamin D₃.

The active vitamin D compounds in accordance with the present invention are valuable for the treatment of breast and colon or colorectal cancer, as well as other neoplasms such as pancreatic cancer, prostate cancer, endometrial cancer, small cell and non-small cell cancer of the lung (including squamous, adenocarcinoma and large cell types), squamous cell cancer of the head and neck, bladder, ovarian and cervical cancers, myeloid and lymphocytic leukemia, lymphoma, hepatic tumors, medullary thyroid carcinoma, multiple myeloma, melanoma, retinoblastoma, and sarcomas of the soft tissue and bone. Concomitant with its value in treatment of hyperproliferative diseases, neoplasms and malignancies, the active vitamin D compounds in accordance with the present invention are beneficial in lowering and/or maintaining lowered PTHrP levels, the elevation or overproduction of which is correlated with hypercalcemia associated with the hyperproliferative diseases.

In accordance with the present invention, when effective amounts of active vitamin D compounds are administered to patients with cancer or neoplasms, the proliferative activity of the abnormal neoplastic cells is inhibited, reduced, or stabilized, and/or cell differentiation is induced, promoted or enhanced. In cases where hypercalcemia is associated with the malignancy, the hypercalcemia is also amelioriated by controlling serum PTHrP levels.

The effective amounts of vitamin D compound are given in an administration protocol of high dosage, generally 10 μg/dose or greater up to 200 μg/dose or greater, given episodically or intermittently. The protocol or dosage regimen in accordance with the present invention provides an improved therapeutic index for active forms of vitamin D analogues compared to administration via conventional regimens. The episodic dosing is also cost effective as less active agent is needed.

Accordingly, another aspect of the invention is a method of treating human cancer comprising administering to a subject who has cancer an effective amount of vitamin D compound which has, or attains through metabolism in vivo, a vitamin D receptor (VDR) binding affinity substantially equivalent to the binding affinity of 1α,25-dihydroxyvitamin D₃ and a hypercalcemia risk substantially lower than that of 1α,25-dihydroxyvitamin D₃ given in known or conventional treatment regimens, to inhibit, decrease or stabilize the cellular abnormal proliferative activity of the cancer. Such hypocalcemic active vitamin D compounds further mitigate the risk of hypercalcemia because of their inherent lower calcemic index.

For treatment for malignant conditions in accordance with the present invention, the active vitamin D compounds of formula (I) can be suitably administered alone as an active ingredient, i.e., as an antiproliferative agent, in a pharmaceutical composition, or co-administered as described hereinbelow with other therapeutic agents, e.g., anticancer (i.e., antiproliferative, cytotoxic, antitumor or antineoplastic) agents. The active vitamin D compound is given in episodic or intermittent high dose. Administration of the active vitamin D may be prior to, simultaneous with, or after administration of the other therapeutic agents.

Specifically included within the scope of the present invention is the co-administration of the active vitamin D of formula (I) with a cytotoxic or anticancer agent; in other words, a combination therapy or treatment. Cytotoxic or antineoplastic agents include antimetabolites, antimicrotubule agents, alkylating agents, platinum agents, anthrocyclines, topisomerase inhibitors, mitotic inhibitors, antibiotics and any other antineoplastic agents such as hormones and hormone antagonists.

It is anticipated that the vitamin D compounds used in combination with various anticancer drugs can give rise to a significantly enhanced cytotoxic or antineoplastic effect on cancerous cells, thus providing an increased therapeutic effect. Specifically, as a significantly increased growth-inhibitory effect is obtained with the above disclosed combinations utilizing lower concentrations of the anticancer drugs compared to the treatment regimes in which the drugs are used alone, there is the potential to provide therapy wherein adverse side effects associated with the various anticancer drugs are considerably reduced compared to side effects normally observed with the anticancer drugs used alone in larger doses. Possible dosage ranges of these co-administered anticancer agents, depending on the nature of the agent, range from about 0.1 to 20 mg/kg which are given on a daily or an episodic or intermittent basis.

Also included within the scope of the present invention is the co-administration of effective dosages of the analogues of formula (I) in conjunction with administration of hormones or other therapeutic agents, e.g., estrogens, which are known to ameliorate bone diseases or disorders. For example, prostate cancer often metastasizes to bone, causing bone loss and associated pain. Such bone agents may include conjugated estrogens or their equivalents, calcitonin, bisphosphonates, calcium supplements, cobalamin, pertussis toxin and boron.

In another aspect, the invention is a pharmaceutical combination which includes an anticancer agent which is an active vitamin D compound and an agent selected from the group consisting of (i) an anticancer (or antineoplastic or antihyperproliferative) agent, (ii) a bone agent, (iii) an antihypercalcemic agent, and combinations thereof. For example, bisphosphonates which have value as bone agents can also be used to mitigate hypercalcemia. Thus, the co-administration of a bisphosphonate with an active vitamin D compound or with an active vitamin D compound and a cytotoxic or antineoplastic agent combination therapy is desirable for further mitigating the risk of hypercalcemia.

All routes of administration of the active vitamin D or its co-administration with other therapeutic agents are suitable. However, parenteral administration of the active vitamin D compounds in accordance with the present invention, alone or in combination with other agents, provides advantages over other treatment modalities. Parenteral administration bypasses the increased calcemic activity that occurs in the gastrointestinal tract from oral administration and reduces incidence or risk of esophagitis. Parenteral dosing also provides for greater compliance and safety because the drugs are generally administered by a health care professional.

Other advantages and a fuller appreciation of specific adaptations, compositional variations, and physical attributes will be gained upon an examination of the following detailed description of preferred embodiments, taken in conjunction with the appended claims.

### BRIEF DESCRIPTION OF THE DRAWINGS

Not applicable.

### DETAILED DESCRIPTION OF THE INVENTION

The present invention provides an effective method for the treatment of neoplasms and other hyperproliferative diseases. Particularly, the present invention relates to therapeutic methods for inhibiting, reducing or stabilizing the hyperproliferative cellular activity of diseased cells (e.g., neoplastic or hyperplastic cells), and inducing, enhancing or promoting cell differentiation in the diseased cells. The present invention provides treatment of a patient suffering from a hyperproliferative disease, such as prostatic cancer or prostatic hyperplasia, with an active vitamin D analogue or compound based on a novel treatment protocol. The active vitamin D compound is suitably a hydroxy vitamin D, e.g., a 1α-hydroxyvitamin D, a 24-hydroxyvitamin D or a 25-hydroxyvitamin D compound. The active vitamin D analogue represented by formula (I) as described hereinbelow is provided to the patient with significantly reduced risk of or without causing dose-limiting hypercalcemia and hypercalciuria, i.e., unphysiologically high and deleterious blood calcium levels and urine calcium levels, respectively. These attributes are achieved through specific chemical properties of the active vitamin D compounds and the novel treatment protocol as described herein.

In accordance with the present invention, when effective amounts of the active vitamin D compounds are administered to patients with cancer or hyperplasia, the proliferative activity of the abnormal cells is inhibited, maintained, or alleviated, and cell differentiation is induced, promoted or enhanced, with significantly less risk of hypercalcemia and hypercalciuria than is observed after the same amount of activated vitamin D₃ is administered in previously known formulations and dosing regimens. The risk of hypercalcemia, long associated with the administration of high doses of vitamin D compounds, is lowered (1) by administering an active vitamin D on an intermittent or episodic basis, especially by administering hypocalcemic active vitamin D compound, or (2) by co-administering the active vitamin D compound an antihypercalcemic agent on an intermittent or episodic basis. Thus, the active vitamin D compounds for use in accordance with the present invention have an improved therapeutic index relative to active forms of vitamin D₃ analogues given in conventional protocols. The treatment protocol in accordance with the present invention provides reduced risk of hypercalcemia, e.g., substantially reduced hypercalcemia; that is, little or no clinical symptoms or signs of hypercalcemia.

It is known that vitamin D₃ must be hydroxylated in the C-1 and C-24 or C-25 positions before it is activated, i.e., before it will produce a biological response. A similar metabolism appears to be required to activate other forms of vitamin D, e.g., vitamin D₂ and vitamin D₄. Therefore, as used herein, the term “activated vitamin D” or “active vitamin D” is intended to refer to a vitamin D compound or analogue that has been hydroxylated in at least one of the C-1, C-24 or C-25 positions of the molecule and either the compound itself or its metabolite in the case of a prodrug, such as 1α-hydroxyvitamin D₂, binds the vitamin D receptor (VDR). For example, vitamin D “prodrugs” or “prohormones” include compounds which are hydroxylated in only one of the three positions. Such compounds undergo further hydroxylation in vivo and their metabolites bind the VDR.

Also, as used herein, the term “lower” as a modifier for alkyl, alkenyl acyl, or cycloalkyl is meant to refer to a straight or branched, saturated or unsaturated hydrocarbon radical having 1 to 4 carbon atoms. Specific examples of such hydrocarbon radicals are methyl, ethyl, propyl, isopropyl, butyl, isobutyl, t-butyl, ethenyl, propenyl, butenyl, isobutenyl, isopropenyl, formyl, acetyl, propionyl, butyryl or cyclopropyl. The term “aromatic acyl” is meant to refer to an unsubstituted or substituted benzoyl group.

As used herein, the term “hydrocarbon moiety” refers to a lower alkyl, a lower alkenyl, a lower acyl group or a lower cycloalkyl, i.e., a straight or branched, saturated or unsaturated C₁-C₄ hydrocarbon radical.

The compound in accordance with the method of the present invention is an active vitamin D compound. The active vitamin D in accordance with the present invention may have an unsaturated sidechain, e.g., there is suitably a double bond between C-22 and C-23, between C-25 and C-26 or between C-25 and C-27.

An active vitamin D of the present invention, i.e., a hydroxyvitamin D, has the general formula described in formula (I) 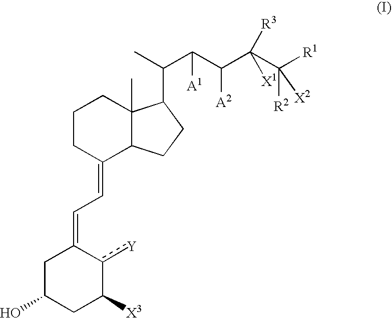
wherein A¹ and A² each are hydrogen or together represent a carbon-carbon bond, thus forming a double bond between C-22 and C-23; R¹ and R² are identical or different and are hydrogen, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that both R¹ and R² cannot both be an alkenyl, or taken together with the carbon to which they are bonded, form a C₃-C₈ cyclocarbon ring; R³ is hydrogen, lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl, X² is hydrogen or hydroxyl, or, may be taken with R¹ or R², to constitute a double bond, X³ is hydrogen or hydroxyl provided that at least one of X¹, X² and X³ is hydroxyl; and Y is a methylene group if the bond to Y is a double bond or is a methyl group or hydrogen if the bond to Y is a single bond.

A 1α-hydroxyvitamin D compound of formula (I) is characterized by the general formula (II): 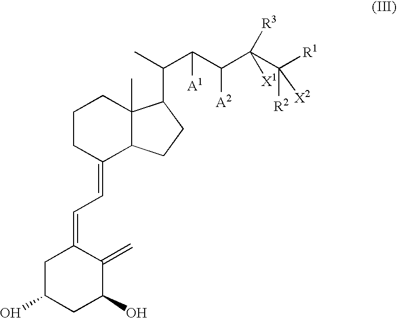
wherein A¹ and A² each are hydrogen or together represent a carbon-carbon bond, thus forming a double bond between C-22 and C-23; R¹ and R² are identical or different and are hydrogen, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that both R¹ and R² cannot both be an alkenyl, or taken together with the carbon to which they are bonded, form a C₃-C₈ cyclocarbon ring; R³ is hydrogen, lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl, X² is hydrogen or hydroxyl, or, may be taken with R¹ or R², to constitute a double bond, and Y is a methylene group if the bond to Y is a double bond or is a methyl group or hydrogen if the bond to Y is a single bond.

Specific 1α-hydroxyvitamin D compounds in accordance with the present invention are characterized by the general formula (III): 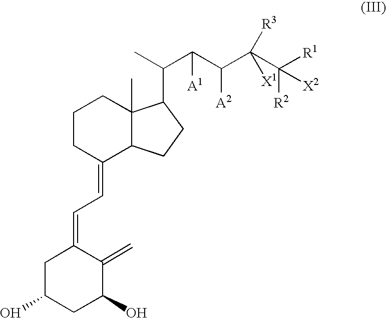
wherein A¹ and A² each are hydrogen or together represent a carbon-carbon bond, thus forming a double bond between C-22 and C-23; R¹ and R² are identical or different and are hydrogen, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that both R¹ and R² cannot both be an alkenyl, or taken together with the carbon to which they are bonded, form a C₃-C₈ cyclocarbon ring; R³ is hydrogen, lower alkyl, lower alkenyl, lower fluoroalkyl, lower fluoroalkenyl, O-lower alkyl, O-lower alkenyl, O-lower acyl, O-aromatic acyl or lower cycloalkyl; X¹ is hydrogen or hydroxyl, and X² is hydrogen or hydroxyl, or, may be taken with R¹ or R², to constitute a double bond. Compounds of formula I, II and III in which Y is hydrogen are also referred to as 19-nor vitamin D compounds.

Specific examples of compounds of formulas (I,) (II) and (III) include, without limitation, 1α,24-dihydroxyvitamin D₂, 1α,24-dihydroxyvitamin D₄, 1α,25-dihydroxyvitamin D₄, 1α,25-dihydroxyvitamin D₂, 1α,24,25-trihydroxyvitamin D₂, 1α,25-dihydroxyvitamin D₃, 1α,24,25-trihydroxyvitamin D₃, and also include such pro-drugs or pro-hormones as 1α-hydroxyvitamin D₂, 1α-hydroxyvitamin D₄, 1α-hydroxyvitamin D₃, 24-hydroxyvitamin D₂, 24-hydroxyvitamin D₄, 24-hydroxyvitaini D₃, 25-hydroxyvitamin D₂, 25-hydroxyvitamin D₄ and 25-hydroxyvitamin D₃. Compounds of particular value are those of formulas (I), (II) and (III) where R³ is not hydrogen, i.e., where R³ is lower alkyl, lower alkenyl, lower fluoroalkyl, lower fluoroalkenyl, O-lower alkyl, O-lower alkenyl, O-lower acyl, O-aromatic acyl or lower cycloalkyl. These are compounds or analogues of vitamin D₂ and vitamin D₄. Of particular value are those vitamin D₂ and D₄ compounds where X¹, X² or X³ are hydroxyl. Such compounds include 1α,24-dihydroxyvitamin D₂, 1α,24-dihydroxyvitamin D₄, 1α,25-dihydroxyvitamin D₄, 1α,25-dihydroxyvitamin D₂, 1α,24,25-trihydroxyvitamin D₂, 1α-hydroxyvitamin D₂, 1α-hydroxyvitamin D₄, 24-hydroxyvitamin D₂, and 24-hydroxyvitamin D₄; they are typically hypocalcemic compared to the natural D hormone, 1α,25-dihydroxyvitamin D₃. By “hypocalcemic” is meant an active vitamin D compound that has reduced calcemic activity compared to that of the natural vitamin D hormone, 1α, 25-dihydroxyvitamin D₃; in other words, a calcemic index less than that of 1α,25-dihydroxyvitamin D₃. “Calcemic index” is a relative measure of the ability of a drug to generate a calcemic response, the calcemic activity of 1α,25-dihydroxyvitamin D₃ being designated as 1. Such hypocalcemia vitamin D compounds provide reduced risk of hypercalcemia even when administered in high doses.

Further, for compounds of formula (I) that have a chiral center, such as at C-24, it is understood that both epimers (e.g., R and S) and the racemic mixture are within the scope of the present invention.

The compounds of formula (I) can be prepared by many widely known methods, e.g., as described, in U.S. Pat. No. 5,488,120 issued to Knutson et al., U.S. Pat. Nos. 4,554,106, 4,670,190 and 5,486,636 issued to DeLuca et al., and Strugnell et al., 310 Biochem. J (1995) pp. 233-241, all of which are incorporated herein by reference.

The present invention provides a method of treating malignant cells as well as other hyperproliferative cells, (i.e., inhibiting their hyperproliferative activity and/or inducing and enhancing their differentiation) with an effective amount of an active vitamin D compound. The effective dosage amount administered to a patient having a hyperproliferative disease is a high dose of active vitamin D compound, including 1α,25-dihydroxyvitamin D₃ (calcitriol), given on an intermittent or episodic dosing regimen. By “high dose” is meant a dose of 10 μg or more, e.g., 20 μg to 100 μg or more, e.g. 200 μg. In other terms, a “high dose” is one that produces in vivo higher than normal physiologic levels of vitamin D, or is sufficient in a single dose to upregulate vitamin D receptors on cells expressing these receptors The intermittent dosing regimen is suitably between once per week to once every 12 weeks, e.g., once every 3 weeks. As a function of body weight, the effective dose ranges from about 0.2 μg to about 3.0 μg per kilogram of body weight of the patient.

Each single dose is sufficient to upregulate vitamin D hormone receptors in target cells. It is believed that continuous dosing is not required because the binding and upregulation by vitamin D compounds is sufficient to initiate the cascade of intracellular metabolic processes occurring with receptor binding. Intermittent dosing reduces the risk of hypercalcemia, and thus, the method in accordance with the present invention can be used to treat hyperproliferative diseases by administering any active vitamin D compound. At the same time, it is contemplated, in accordance with the present invention, that the risk of hypercalcemia can be further mitigated if the active vitamin D compound is a hypocalcemic active vitamin D compound.

The compounds of the present invention given in the illustrated dosing regimen, thus, overcome the shortcomings of the known active vitamin D₃ compounds described above, and can be considered preferred agents for the control and treatment of malignant diseases such as breast, prostate, testicular and colon or colorectal cancer, as well as other neoplasms such as pancreatic cancer, endometrial cancer, small cell and non-small cell cancer of the lung (including squamous, adneocarcinoma and large cell types), squamous cell of the head and neck, bladder, ovarian and cervical cancers, myeloid and lymphocyltic leukemia, lymphoma, hepatic tumors, medullary thyroid carcinoma, multiple myeloma, melanoma, retinoblastoma, and sarcomas of the soft tissue and bone, i.e. neoplasms that express a vitamin D receptor. Hyperproliferative conditions that may be treated by the method of the present invention also include psoriasis and hyperplasias such as prostate hyperplasia.

It is further believed that the intermittent high dose regimen can be used to effect any therapeutic effect that is attributable to active vitamin D., e.g., antiproliferative activity, reduction of loss of bone mass, etc. In regard to antiproliferative activity, the value of the intermittent dosing is that antihyperproliferative activity and upregulation of vitamin D receptors occurs with a single dose without the side effects of hypercalcemia and hypercalciuria that occur with recurrent daily dosing. At the same time, the intermittent dosing regimen is also sufficient to control levels of parathyroid hormone related protein (e.g., potentially by downregulation of expression of PTHrP).

The episodic dose can be a single dose or, optionally, divided into 2-4 subdoses which, if desired, can be given, e.g., twenty minutes to an hour apart until the total dose is given. The compounds in accordance with the present invention are administered in an amount that raises serum vitamin D levels to a supraphysiological level for a sufficient period of time to induce differentiation or regression of a tumor or neoplasm without causing hypercalcemia or with substantially reduced the risk of hypercalcemia. The properties of the hypocalcemic vitamin D compounds are particularly beneficial in permitting such supraphysiologic levels.

The pharmacologically active compounds of this invention can be processed in accordance with conventional methods of pharmacy to produce medicinal agents for administration to patients, e.g., mammals including humans. For example, the active vitamin D compounds of the present invention can be formulated in pharmaceutical compositions in a conventional manner using one or more conventional excipients, which do not deleteriously react with the active compounds, e.g., pharmaceutically acceptable carrier substances suitable for enteral administration (e.g., oral), parenteral, topical, buccal or rectal application, or by administration by inhalation or insufflation (e.g., either through the mouth or the nose)

Generally, acceptable carriers for pharmaceutical formulation include, but are not limited to, water, salt solutions, alcohols, gum arabic, vegetable oils (e.g., almond oil, corn oil, cottonseed oil, peanut oil, olive oil, coconut oil), mineral oil, fish liver oils, oily esters such as Polysorbate 80, polyethylene glycols, gelatine, carbohydrates (e.g., lactose, amylose or starch), magnesium stearate, talc, silicic acid, viscous paraffin, fatty acid monoglycerides and diglycerides, pentaerythritol fatty acid esters, hydroxy methylcellulose, polyvinyl pyrrolidone, etc.

Of particular interest is the parenteral, e.g., injectable, dosage form. Using the parenteral route of administration allows for bypass of the first pass of active vitamin D compound through the intestine, thus avoiding stimulation of intestinal calcium absorption, and further reduces the risk of esophageal irritation which is often associated with high dose oral administration. Because an injectable route of administration is typically done by a health care professional, the dosing can be more effectively controlled as to precise amount and timing. Parenteral administration suitably includes subcutaneous, intramuscular, or intravenous injection, nasopharyngeal or mucosal absorption, or transdermal absorption. Where indicated, the compounds of formula (I) may also be given by direct injection into the tumor, e.g., a parathyroid adenoma, or by regional delivery, e.g., by intraarterial delivery or delivery via the portal vein. Regional delivery is especially suitable for treatment of hepatic cancers.

The injectable compositions may take such forms as sterile suspensions, solutions, or emulsions in oily vehicles (such as coconut oil, cottonseed oil, sesame oil, peanut oil or soybean oil) or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically sterile, pyrogen-free water, saline, aqueous propylene glycol, or another injectable liquid, e.g., peanut oil for intramuscular injections. Also, various buffering agents, preservatives, suspending, stabilizing or dispensing agents, surface-active agents and the like can be included. Aqueous solutions may be suitably buffered, if necessary, and the liquid diluent first rendered isotonic with sufficient saline or glucose. Aqueous solutions are especially suitable for intravenous, intramuscular, subcutaneous and intraperitoneal injection purposes. In this connection, the sterile aqueous media employed are all readily obtainable by standard techniques well-known to those skilled in the art. The oily solutions are suitable for intra-articular, intramuscular and subcutaneous injection purposes. The preparation of all these solutions under sterile conditions is readily accomplished by standard pharmaceutical techniques well-known to those skilled in the art. Additionally, it is also possible to administer the compounds of the present invention topically when treating pathological conditions of the skin, and this may suitably be done by way of creams, jellies, gels, pastes, ointments and the like, in accordance with standard pharmaceutical practice.

The compounds formulated for parenteral administration by injection may be administered, by bolus injection or continuous infusion. Formulations for injection may be conveniently presented in unit dosage form, e.g., in ampoules or in multi-dose containers, with an added preservative.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, e.g., a sparingly soluble salt.

Although it is considered that episodic parenteral administration of high dose active vitamin D is highly beneficial, it is also contemplated within the scope of the present invention that enteral dosing, e.g., oral administration, can also be of benefit. Thus, episodic enteral dosing of high dose active vitamin D is also considered of benefit in achieving the upregulation of cell receptors and control of PTHrP in treatment of hyperproliferative diseases.

For enteral application, particularly suitable are tablets, dragees, liquids, drops, suppositories, lozenges, powders, or capsules. A syrup, elixir, or the like can be used if a sweetened vehicle is desired. For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may also be suitably formulated to give controlled release of the active compound. Many controlled release systems are known in the art.

For buccal administration, the compositions may take the form of tablets, lozenges or absorption wafers formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of e.g. gelatin, for use in an inhaler or insufflator may be formulated containing a powder mix of the active compound and a suitable powder base such as lactose or starch.

The compounds may also be formulated in rectal or vaginal compositions such as suppositories containing conventional suppository bases or retention enemas. These compositions can be prepared by mixing the active ingredient with a suitable non-irritating excipient which is solid at room temperature (for example, 10° C. to 32° C.) but liquid at the rectal temperature, and will melt in the rectum or vagina to release the active ingredient. Such materials are polyethylene glycols, cocoa butter, other glycerides and wax. To prolong storage life, the composition advantageously may include an antioxidant such as ascorbic acid, butylated hydroxyanisole or hydroquinone.

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration.

For topical application, suitable nonsprayable viscous, semi-solid or solid forms can be employed which include a carrier compatible with topical application and having a dynamic viscosity preferably greater than water, for example, mineral oil, almond oil, self-emulsifying beeswax, vegetable oil, white soft paraffin, and propylene glycol. Suitable formulations include, but are not limited to, creams, ointments, lotions, solutions, suspensions, emulsions, powders, liniments, salves, aerosols, transdermal patches, etc., which are, if desired, sterilized or mixed with auxiliary agents, e.g., preservatives, stabilizers, demulsifiers, wetting agents, etc. A cream preparation in accordance with the present invention suitably includes, for example, mixture of water, almond oil, mineral oil and self-emulsifying beeswax; an ointment preparation suitably includes, for example, almond oil and white soft paraffin; and a lotion preparation suitably includes, for example, dry propylene glycol. For purposes of transdermal administration, dilute sterile, aqueous or partially aqueous solutions (usually in about 0.1% to 5% concentration), otherwise similar to the above parenteral solutions, are prepared.

Topical preparations of the compounds in accordance with the present invention useful for the treatment of pathological skin disorders may also include epithelialization-inducing agents such as retinoids (e.g., vitamin A), chromanols such as vitamin E, β-agonists such as isoproterenol or cyclic adenosine monophosphate (cAMP), anti-inflammatory agents such as corticosteroids (e.g., hydrocortisone or its acetate, or dexamethasone) and keratoplastic agents such as coal tar or anthralin. Effective amounts of such agents are, for example, vitamin A about 0.003 to about 0.3% by weight of the composition; vitamin E about 0.1 to about 10%; isoproterenol about 0.1 to about 2%; cAMP about 0.1 to about 1%; hydrocortisone about 0.25 to about 5%; coal tar about 0.1 to about 20%; and anthralin about 0.05 to about 2%.

The pharmaceutical preparations can be sterilized and, if desired, be mixed with auxiliary agents, e.g., lubricants, preservatives, stabilizers, wetting agents, emulsifiers, salts for influencing osmotic pressure, buffers, coloring, flavoring and/or one or more other active compounds, for example, conjugated estrogens or their equivalents, anti-estrogens, calcitonin, bisphosphonates, calcium supplements, cobalamin, pertussis toxin, boron, antineoplastic agents and antihypercalcemic agents.

Since the present invention has an aspect that relates to inhibition of proliferation of malignant or neoplastic cells by treatment with a combination of active ingredients which may be administered separately, the invention also relates to combining separate pharmaceutical compositions in kit form. The kit includes two or more separate pharmaceutical compositions: a compound of Formula (I) and one or more other agents as described hereinbelow. The kit suitably includes container means for containing the separate compositions such as a divided bottle or a divided foil packet. Typically, the kit includes directions for the administration of the separate components. The kit form is particularly advantageous when the separate components are administered in different dosage forms (e.g., oral and parenteral), are administered at different dosage intervals, or when titration of the individual components of the combination is desired by the prescribing physician.

The dosage of the compounds for the treatment of cancer or neoplasms with the active vitamin D compounds in accordance with the present invention can be done on an episodic basis, in which high doses can be used, generally about 10 μg to about 200 μg, e.g., 10 μg to 100 μg, given once per week to up to once every 12 weeks, typically orally or parenterally, although other routes can be used as described herein. Generally, the compounds of this invention are dispensed by unit dosage form in a pharmaceutically acceptable carrier as described herein.

For topical treatment of skin disorders, (e.g. psoriasis) the dosage of the compound of the present invention in a topical composition generally is about 0.01 μg to about 50 μg per gram of composition. For treatment of skin cancers, the dosage of the vitamin D compound in a locally applied composition generally is about 0.01 μg to 100 μg per gram composition.

Those of ordinary skill in the art will readily optimize effective doses and co-administration regimens (as described hereinbelow) as determined by good medical practice and the clinical condition of the individual patient. Regardless of the manner of administration, it will be appreciated that the actual preferred amounts of active compound in a specific case will vary according to the efficacy of the specific compound employed, the particular compositions formulated, the mode of application, and the particular situs and organism being treated. For example, the specific dose for a particular patient depends on age, body weight, general state of health, on diet, on the timing and mode of administration, on the rate of excretion, and on medicaments used in combination and the severity of the particular disorder to which the therapy is applied. Dosages for a given patient can be determined using conventional considerations, e.g., by customary comparison of the differential activities of the subject compounds and of a known agent, such as by means of an appropriate conventional pharmacological protocol. A physician of ordinary skill can readily determine and prescribe the effective amount of the drug required to counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug. The dosage of active ingredient in the compositions of this invention may be varied; however, it is necessary that the amount of the active ingredient be such that an efficacious dosage is obtained. The active ingredient is administered to patients (animal and human) in need of treatment in dosages that will provide optimal pharmaceutical efficacy.

Further, included within the scope of the present invention is a method of co-administration of active vitamin D compounds with an anticancer or antineoplastic agent. In accordance with the present invention, therapeutic antihyperproliferative benefits are achieved with intermittent dosing of active vitamin D with cytotoxic, i.e., other chemotherapeutic or antineoplastic, agents. Many antineoplastic or cytotoxic agents must be delivered through a parenteral route of administration, and thus, a protocol of injectable active vitamin D and antineoplastic agent can be set up on a routine basis. The co-administration of active vitamin D and antineoplastic agents can be prior to, after, or simultaneous with each other. However, it is believed that the prior administration of active vitamin D with the later episodic administration of a cytotoxic or antineoplastic agent is of benefit. For example, the high dose active vitamin D upregulates the receptors, and primes and promotes cell differentiation. Such upregulation and priming, potentially permits less cytotoxic or antineoplastic agent than would typically be required if the cytotoxic agent were administered alone.

The term “co-administration” is meant to refer to a combination therapy by any administration route in which two or more agents are administered to a patient or subject. Co-administration of agents may be referred to as combination therapy or combination treatment. The agents may be the same dosage formulations or separate formulations. For combination treatment with more than one active agent, where the active agents are in separate dosage formulations, the active agents can be administered concurrently, or they each can be administered at separately staggered times. The agents may be administered simultaneously or sequentially, as along as they are given in a manner sufficient to allow both agents to achieve effective concentrations in the body. The agents may be administered by different routes, e.g., one agent may be administered intravenously while a second agent is administered intramuscularly, intravenously or orally. The agents also may be in an admixture, as, for example, in a single tablet.

In time-sequential co-administration, one agent may directly follow administration of the other or the agents may be give episodically, i.e., one can be given at one time followed by the other at a later time, e.g., within a week. An example of a suitable co-administration regimen is where an active vitamin D compound is administered from 0.5 to 7 days prior to administration of a cytotoxic or antineoplastic agent.

Cytotoxic or antineoplastic agents include antimetabolites (mitotic inhibitors), antimicrotubule agents, alkylating agents, platinum agents, anthrocyclines, topisomerase inhibitors, antibiotics and other agents such as hormones and antagonists. The antimetabolites include pyrimidine and purine analogs and inhibitors, such as 5-fluorouracil, floxuridine, cytarabine, mercaptopurine, thioguanine, pentostatin, cladribine and fludarabine, and folic acid analogs, such as methotrexate. The antimicrotubule agents include vincristine, vinblastine and taxanes such as paclitaxel and docetaxel. The alkylating agents include nitrogen mustards, such as mechlorethamine, cyclophosphamide, ifosfamide, melphalan, chlorambucil, alkyl sulfonates such as busulfan, nitrosoureas such as carmustine, lomustine, and streptozocin as well as other agents such as hexamethylmelamine, thiotepa, dacarbozine and temozolomide. The platinum agents include cisplatin, carboplatin, oxaliplatin, JM-216, and CI-973. The anthracylines include doxorubicin and daunorubicin. The topoisomerase inhibitors include etoposide, teniposide, the camptothecens such as toptecan and irinotecan. The antibiotics include mitomycin, andriamycin, dactinomycin, daunomycin, idarabicin and bleomycin. Other chemotherapeutic agents include hormones such as adrenocorticosteroids (e.g. prednisone), progestins (e.g., hydroxyprogesterone caproate, medroxyprogesterone acetate, megestrol acetate), estrogens (e.g., diethylstibestrol, ethinyl estrodiol), antiestrogens (e.g., tamoxifen, anastrozole), androgens (e.g., testosterone propionate, fluoxymesterone), antiandrogen (e.g., flutamide) and gonado-releasing hormone analogs (e.g., leuprolide). Still other agents useful in neoplastic disease are biological response modifiers (e.g., interferon-alpha interleuben-2), anthracenediones (e.g., mitoanthrone), substituted ureas (e.g. hydroxyurea), methylhydrozine derivatives (e.g. procarbazine) adrenocorticol suppressants (e.g. mitotane, aminoglutethimide), tyrosine kinase inhibitors (e.g. imatinib), enzymes (e.g. L-asparagine), as well as estramustine phosphate and prednimustine.

It is anticipated that active vitamin D compounds used in combination with various anticancer drugs can give rise to a significantly enhanced cytotoxic or antineoplastic effect on cancerous or neoplastic cells, thus providing an increased therapeutic effect. Specifically, as a significantly increased growth-inhibitory effect is obtained with the above disclosed combinations utilizing lower concentrations of the anticancer drugs compared to the treatment regimes in which the drugs are used alone, there is the potential to provide therapy wherein adverse side effects associated with the anticancer drugs are considerably reduced compared to side effects normally observed with the anticancer drugs used alone in larger doses. Possible dose ranges of these co-administered anticancer agents are about 0.1 to 20 mg/kg/day or similar dosage amounts.

Also included within the scope of the present invention is the co-administration of effective dosages of active vitamin D compounds with hormones or other agents, e.g., estrogens, that are known to ameliorate bone diseases or disorders. For example, prostate cancer often metastasizes to bone, causing bone loss and associated pain. Such bone agents may include conjugated estrogens or their equivalents, calcitonin, bisphosphonates, calcium supplements, cobalamin, pertussis toxin and boron. Possible dose ranges for these co-administered bone agents are provided in Table 1.

TABLE 1
Possible Oral Dose Ranges for Various Bone Agents Co-Administered
With 1α-Hydroxyvitamin D of Formula (I)

| | Dose Ranges | | |
|---|---|---|---|
| Agent | Broad | Preferred | Most Preferred |
| Conjugated Estrogens or | 0.3-5.0 | 0.4-2.4 | 0.6-1.2 |
| Equivalent (mg/day) | | | |
| Sodium Fluoride (mg/day) | 5-150 | 30-75 | 40-60 |
| Calcitonin (IU/day) | 5-800 | 25-500 | 50-200 |
| Bisphosphonates (mg/day) | 0.5-20 | 1-15 | 5-10 |
| Calcium Supplements | 250-2500 | 500-1500 | 750-1000 |
| (mg/day) | | | |
| Cobalamin (μg/day) | 5-200 | 20-100 | 30-50 |
| Pertussis Toxin (mg/day) | 0.1-2000 | 10-1500 | 100-1000 |
| Boron (mg/day) | 0.10-3000 | 1-250 | 2-100 |

Antiestrogens, such as Tamoxifen™, are also known bone agents and may be suitably used in conjunction with the active D compounds of the present invention.

It is further noted that certain bone agents and cytotoxic agents may also be of value because of their antihypercalcemic properties, and thus can be co-administered on an episodic basis in accordance with the present invention to further reduce the risk of hypercalcemia. Such agents include the bisphosphonates such as pamidronate, and cytotoxic agents such as mitomycin. Antihypercalcemic agents may also include corticosteriods.

Combinations of these therapeutic agents, some of which have also been mentioned herein, with an active vitamin D compound will bring additional, complementary, and often synergistic properties to enhance the desirable properties of these various therapeutic agents. In such combination therapy, the active vitamin D compound may be administered with the other therapeutic agent (e.g., concurrently, concombinantly, sequentially, or in a unitary formulation) such that their therapeutic efficacy overlap

The present invention is further explained by the following examples which should not be construed by way of limiting the scope of the present invention. α

### VDR Binding Analyses

### EXAMPLE 1

### 1α,24-dihydroxyvitamin D₂ [1α,24-(OH)₂D₂]

VDR binding of vitamin D compounds by prostate cells is demonstrated using the techniques of Skowronski et al., 136 Endocrinology (1995) 20-26, which is incorporated herein by reference. Prostate-derived cell lines are cultured to near confluence, washed and harvested by scraping. Cells are washed by centrifugation, and the cell pellet resuspended in a buffered salt solution containing protease inhibitors. The cells are disrupted by sonication while cooling on ice. The supernatant obtained from centrifuging the disrupted cells at 207,000×g for 35 min at 4° C. is assayed for binding. 200 μL of soluble extract, (1-2 mg protein/ml supernatant) is incubated with 1 nM ³H-1α,25-(OH)₂D₃ and increasing concentrations of 1α,24-(OH)₂-D₂ (0.01-100 nM) for 16-20 hr at 4 EC. Bound and free hormones are separated with hydroxyapatite using standard procedures. Specific binding is calculated by subtracting nonspecific binding obtained in the presence of a 250-fold excess of nonradioactive 1α,25-(OH)₂D₃ from the total binding measured. The results demonstrate that 1α,24-(OH)₂D₂ has strong affinity for prostate VDR, indicating that 1α,24-(OH)₂D₂ has potent biological activity in respect of prostate cells.

### EXAMPLE 2

### 1α,24-dihydroxy vitamin D₄ [1α,24-(OH)₂D₄]

The procedure of Example 1 is repeated using the active vitamin D analogue 1α,24-(OH)₂D₄, and the specific binding is determined. The results demonstrate that 1α,24-(OH)₂D₄ has strong affinity for prostate VDR, indicating that 1α,24-(OH)₂D₄ has potent biological activity in respect of prostate cells.

### EXAMPLE 3

### 1α,25-dihydroxyvitamin D₄ [1α,25-(OH)₂D₄]

The procedure of Example 1 is repeated using the active vitamin D analogue 1α,25-(OH)₂D₄, and the specific binding is determined. The results demonstrate that 1α,25-(OH)₂D₄ has strong affinity for prostate VDR, indicating that 1α,25-(OH)₂D₄ has potent biological activity in respect of prostate cells.

### Gene Expression

### EXAMPLE 4

### 1α,24-dihydroxy vitamin D₄ [1α,24-(OH)₂D₄]

Using the plasmids p(CT4)⁴TKGH, a vitamin D receptor (VDR)-expressing plasmid, and pSG5-hVDR1/3, a plasmid containing growth hormone (GH) gene under the control of a vitamin D-responsive element (VDRE), experiments were conducted to explore the ability of 1α,24-(OH)₂D₄ to induce vitamin D-dependent growth hormone acting as a reporter gene compared to that of 1α,25-(OH)₂D₃. Cells in culture were transfected with these two plasmids. These transfected cultures were incubated with 1α,24-(OH)₂D₄ or 1α,25-(OH)₂D₃, and the production of growth hormone was measured. Table 2 below shows the results of this assay:

TABLE 2
Induction of Growth Hormone by Vitamin D Compounds

| | Concentration | Growth Hormone |
|---|---|---|
| Compound | Used (M) | Induction (ng/ml) |
| 1,25-(OH)₂D₃ | 1 × 10⁻¹⁰ | 39 |
| 1,25-(OH)₂D₃ | 5 × 10⁻¹⁰ | 248 |
| 1,24-(OH)₂D₄ | 5 × 10⁻¹⁰ | 165 |
| 1,24-(OH)₂D₄ | 1 × 10⁻⁹ | 628 |
| 1,24-(OH)₂D₄ | 5 × 10⁻⁹ | 1098 |

These data show that the ability of 1α,24-(OH)₂D₄ to stimulate vitamin D-dependent growth hormone is nearly equivalent to that of 1α,25-(OH)₂D₃. Such results are truly surprising and would not have been expected by following the teachings of the prior art.

### EXAMPLE 5

### 1α,24(S)-dihydroxyvitamin D₂ and 1α,24(R)-dihydroxy-vitamin D₂ [1α,24(S)-(OH)₂D₂ and 1α,24(R)-(OH)₂D₂]

The gene expression study described in Example 4 was conducted to compare the biological activity in vitro of chemically synthesized 1α,24(S)-(OH)₂D₂ and 1α,24(R)-(OH)₂D₂, with 1α,25-(OH)₂D₃ and 25-OH-D₃. The vitamin D-dependent transcriptional activation model system was used in which plasmids pSG5-hVDR1/3 and p(CT4)⁴TKGH were co-transfected into Green monkey kidney, (COS-1) cells.

Transfected cells were incubated with vitamin D metabolites and growth hormone production was measured. As shown in Table 3, both 1α,24(S)-(OH)₂D₂ and its epimer, 1α,24(R)-(OH)₂D₂, had significantly more activity in this system than 25-OH-D₃, with 1α,24(S)-(OH)₂D₂ having nearly the same activity as 1α,25-(OH)₂D₃.

TABLE 3
Vitamin D-Inducible Growth Hormone Production
In Transfected COS-1 Cells

| | Vitamin D Inducible Growth Hormone Production | | |
|---|---|---|---|
| | | | Net vitamin |
| | | Total GH | D inducible |
| | Molar | Production* | GH-production |
| Inducer | Concentration | (ng/ml) | (ng/ml) |
| Ethanol | | 44 | 0 |
| 25-OH-D₃ | 1 × 10⁻⁷ | 245 | 201 |
| | 1 × 10⁻⁶ | 1100 | 1056 |
| | 1 × 10⁻⁵ | 775 | 731 |
| 1α,25-(OH)₂D₃ | 1 × 10⁻¹⁰ | 74 | 30 |
| | 1 × 10⁻⁹ | 925 | 881 |
| | 1 × 10⁻⁸ | 1475 | 1441 |
| 1α,24(S)—(OH)₂D₂ | 5 × 10⁻¹⁰ | 425 | 381 |
| | 5 × 10⁻⁹ | 1350 | 1306 |
| | 5 × 10⁻⁸ | 1182 | 1138 |
| 1α,24(R)—(OH)₂D₂ | 1 × 10⁻⁹ | 80 | 36 |
| | 1 × 10⁻⁸ | 1100 | 1056 |
| | 1 × 10⁻⁷ | 1300 | 1256 |

*Averages of duplicate determinations

### Inhibition of Cell Proliferation

### EXAMPLE 6

### 1α,24-dihydroxyvitamin D₂ [1α,24-(OH)₂D₂]

Inhibition of cell proliferation is demonstrated using the techniques of Skowronski et al., 132 Endocrinology (1993) 1952-1960 and 136 Endocrinology (1995) 20-26, both of which are incorporated herein by reference. The cell lines, LNCaP and PC-3, which are derived from human prostate adenocarcinoma, are seeded in six-well tissue culture plates at a density of about 50,000 cells/plate. After the cells have attached and stabilized, about 2-3 days, the medium is replenished with medium containing vehicle or the active vitamin D analogue 1α,24-(OH)₂D₂, at concentrations from 10⁻¹¹ M to 10⁻⁷ M. Medium containing test analogue or vehicle is replaced every three days. After 6-7 days, the medium is removed, the cells are rinsed, precipitated with cold 5% trichloroacetic acid, and washed with cold ethanol. The cells are solubilized with 0.2 N sodium hydroxide, and the amount of DNA determined by standard procedures. The results show that cultures incubated with 1α,24-(OH)₂D₂ in accordance with the present invention have significantly fewer cells than the control cultures.

### EXAMPLE 7

### 1α,24-dihydroxy vitamin D₄ [1α,24-(OH)₂D₄]

The procedure of Example 6 is repeated using the active vitamin D compound 1α,24-(OH)₂D₄, and the cell number is determined. Cultures incubated with 1α,24-(OH)₂D₄ have significantly fewer cells than the control cultures.

### EXAMPLE 8

### 1α,25-dihydroxyvitamin D₄ [1α,25-(OH)₂D₄]

The procedure of Example 6 is repeated using the active vitamin D compound 1α,25-(OH)₂D₄, and the cell number is determined. Cultures incubated with 1α,25-(OH)₂D₄ have significantly fewer cells than the control cultures.

### Stimulation of Cell Differentiation

### EXAMPLE 9

### 1α,24-dihydroxyvitamin D₂ [1α,24-(OH)₂D₂]

Using the techniques of Skowronski et al., 132 Endocrinology (1993) 1952-1960 and 136 Endocrinology (1995) 20-26, both of which are incorporated herein by reference, cells of the cell line, LNCaP, which is derived from a human metastatic prostate adrenocarcinoma and known to express PSA, are seeded in six-well tissue culture plates at a density of about 50,000 cells/well in 3 mL RPMI-1640 containing 5% FBS. After the cells have attached and stabilized, about 2-3 days, the medium is replenished with medium containing vehicle or the active vitamin D analogue, 1α,24-(OH)₂D₂, at concentrations from 10⁻¹¹ M to 10⁻⁷ M. After 6-7 days, the medium is removed and stored at −20° C. for prostate specific antigen (PSA) analysis. PSA in these studies is used as a marker for cellular differentiation.

The cells from parallel cultures are rinsed, precipitated, and the amount of DNA determined by standard procedures. PSA is measured by standard known methods. Cultures incubated with 1α,24-(OH)₂D₂ have significantly more PSA than control cultures when expressed as mass of PSA/cell.

### EXAMPLE 10

### 1α,24-dihydroxyvitamin D₄ [1α,24-(OH)₂D₄]

The procedure of Example 9 is repeated except the active vitamin D compound is 1α,24-(OH)₂D₄. The PSA is measured and cultures incubated with 1α,24-(OH)₂D₄ have significantly more PSA than control cultures when expressed as mass of PSA/cell.

### EXAMPLE 11

### 1α,25-dihydroxyvitamin D₄ [1α,24-(OH)₂D₄]

The procedure of Example 9 is repeated except the active vitamin D compound is 1α,25-(OH)₂D₄. The PSA is measured and cultures incubated with 1α,25-(OH)₂D₄ have significantly more PSA than control cultures when expressed as mass of PSA/cell.

### Clinical Studies

### EXAMPLE 12

### General Treatment of Cancers

Patients with a known vitamin D receptor positive tumor (e.g., adenocarcinoma of the prostate, breast, lung, colon or pancreas, or transitional cell carcinoma of the bladder, or melanoma) participate in an open-label study of an active vitamin D compound in accordance with the present invention. Patients are placed on a reduced calcium diet prior to treatment, to help minimize intestinal absorption and allow ever higher doses of the active vitamin D. This reduced calcium diet may be continued for the duration of treatment, and for one week after the last dose of the active vitamin D. The diet ideally restricts daily calcium intake to 400-500 mg. Patients also discontinue use of any vitamin D supplements or vitamin D replacement therapies. Each patient is also asked to drink 4-6 cups of fluid more than usual intake to assure adequate oral hydration.

Each subject is monitored at regular intervals for: (1) hypercalcemia, hyperphosphatemia, hypercalciuria, hyperphosphaturia and other toxicity; (2) evidence of changes in the progression of metastatic disease; and (3) compliance with the prescribed test drug dosage.

A non-daily, episodic dosing regimen is used, e.g., 10 μg or 20 μg per dose to about 100 μg or 200 μg/dose given once a week to once every 12 weeks. The route of administration can vary from oral to intravenous to regional delivery (e.g., arterial infusion, via the portal vein). Oral is typically the easiest route; however, intravenous administration is advantageous for high dosing because, for example, it generally avoids hypercalcemia due to stimulation of calcium absorption in the intestine. Regional delivery also permits high dosing and generally avoids any hypercalcemia. Although, in the case of the hypocalcemic compounds of the present invention, these compounds are inherently of low risk of producing hypercalcemia.

After 18 months of treatment, CAT scans, X-rays and bone scans used for evaluating the progress of metastatic disease show stable disease and partial or complete remission in many patients treated at the high dosage episodic regimen.

### EXAMPLE 13

### Treatment of prostate cancer with 1α,24-dihydroxy vitamin D₂ [1α,24-(OH)₂D₂]

Patients with advanced androgen-independent prostate cancer participate in an open-label study of 1α,24-(OH)₂D₂. Qualified patients are at least 40 years old, exhibit histologic evidence of adenocarcinoma of the prostate, and present with progressive disease which had previously responded to hormonal intervention(s). On admission to the study, patients begin a course of therapy with oral or intravenous 1α,24-(OH)₂D₂ lasting 26 weeks, while discontinuing any previous use of calcium supplements, vitamin D supplements, and vitamin D hormone replacement therapies. During treatment, the patients are monitored at regular intervals for: (1) hypercalcemia, hyperphosphatemia, hypercalciuria, hyperphosphaturia and other toxicity; (2) evidence of changes in the progression of metastatic disease; and (3) compliance with the prescribed test drug dosage.

The study is conducted in two phases. During the first phase, a maximal tolerated dosage of intravenous 1α,24-(OH)₂D₂ is determined by administering progressively higher dosages to successive groups of patients. The first group of patients is treated with 25.0 μg of 1α,24-(OH)₂D₂. Subsequent groups of patients are treated with 50.0, 75.0 and 100.0 μg/day, all administered once per week. Dosing is continued uninterrupted for the duration of the study unless serum calcium exceeds 11.6 mg/dL, or other toxicity of grade 3 or 4 is observed, in which case dosing is held in abeyance until resolution of the observed toxic effect(s) and then resumed at a level which has been decreased by 10.0 μg.

Results from the first phase of the study show that episodic doses for 1α,24-(OH)₂D₂ which are above 20.0 μg/day, a level which is 10- to 40-fold higher than can be achieved with 1α,25-(OH)₂D₃ are well tolerated with little clinical symptoms of hypercalcemia. Analysis of blood samples collected at regular intervals from the participating patients reveal that the levels of circulating 1α,24-(OH)₂D₂ increase proportionately with the dosage administered, rising to maximum levels well above 100 pg/mL at the highest dosages, and that circulating levels of 1α,25-(OH)₂D₃ are suppressed, often to undetectable levels. Serum and urine calcium are elevated in a dose responsive manner. Patients treated with the maximum tolerated dose of 1α,24-(OH)₂D₂ for at least six months report that bone pain associated with metastatic disease is significantly diminished.

During the second phase, patients are treated with 1α,24-(OH)₂D₂ for 24 months with 20 μg and 100 μg/dose given once per week. After one and two years of treatment, CAT scans, X-rays and bone scans used for evaluating the progression of metastatic disease show stable disease or partial remission in many patients treated at the lower dosage, and stable disease and partial or complete remission in many patients treated at the higher dosage.

### EXAMPLE 14

### Treatment of Prostate Cancer with 1α-hydroxyvitamin D₂ [1α-OH-D₂]

The study of Example 13 is repeated for the active vitamin D compound, 1α-OH-D₂. The results of the phase one study indicate that patients treated with the 20 μg of 1α-OH-D₂ once per week for at least six months report that bone pain associated with metastatic disease is significantly diminished. The results of the phase two study indicate that after two years, CAT scans, X-rays and bone scans used for evaluating the progression of metastatic disease show stable disease or partial or complete remission in many patients.

### EXAMPLE 15

### Treatment of Melanoma

The method of Example 13 is used to treat patients with metastatic malignant melanoma of, e.g., the jaw. After 18 months of treatment, the progress of the metastatic disease shows stable disease or partial remission.

### EXAMPLE 16

### Treatment of Retinoblastoma

The method of Example 13 is used is used to treat patients with metastatic retinoblastoma. After 18 months of treatment, the progress of the metastatic disease shows stable disease or partial remission.

### EXAMPLE 17

### Treatment of Liver Cancer

The method of Example 13 is used to treat patients with hepatoma. The regional delivery of the compound in accordance with the present invention, i.e., via arterial infusion, is used. After 18 months of treatment, the progress of the metastatic disease shows stable disease or partial remission.

### EXAMPLE 18

### Treatment of Cancer by Episodic Co-Administration of Active Vitamin D and an Antineoplastic Agent

Patients with malignant tumors participate in a treatment regimen of 1α,24-(OH)₂D₂ and paclitaxel. Both the active vitamin D and paclitaxel are given intravenously. Paclitaxel is given in a 3-hour infusion, once every 3 weeks with the active vitamin D co-administered once every 3 weeks for 26 weeks. The dosage of paclitaxel is 80 mg/m² and the 1α,24-(OH)₂D₂ is 50 μg/dose.

Patients that complete the regimen are evaluated. No toxicity is observed in any patients. The progress of the tumors shows stable disease or partial or complete remission.

### EXAMPLE 19

### Treatment of Cancer by Co-Administration of Active Vitamin D Compound, an Antineoplastic Agent and an Antihypercalcemic Agent

The method of Example 18 is used to treat patients with malignant tumors by a treatment regimen that includes an antihypercalcemic agent as well as the active vitamin D and the antineoplastic agent. The treatment regimen includes, e.g., 1α-OH-D₂, 1α,24-(OH)₂D₂ or 1α,25-(OH)₂D₃, paclitaxel and pamidronate. All active agents are co-administered once every three weeks for 26 weeks.

The patients completing the treatment regimen are evaluated and show serum calcium levels that do not exceed 11.6 mg/dL and exhibit no clinical symptoms such as dehydration and cachexia attributable to hypercalcemia. These results demonstrate that pamidronate significantly reduces the risk of hypercalcemia in an antineoplastic treatment regimen.

While the present invention has now been described and exemplified with some specificity, those skilled in the art will appreciate the various modifications, including variations, additions, and omissions, that may be made in what has been described. Accordingly, it is intended that these modifications also be encompassed by the present invention and that the scope of the present invention be limited solely by the broadest interpretation lawfully accorded the appended claims.

1. A method of inhibiting hyperproliferation of malignant or neoplastic cells, comprising treating the cells episodically with an antiproliferative amount of an active vitamin D compound; the cells expressing a vitamin D receptor, wherein the amount of active vitamin D is a high dose which is between about 10 μg to about 200[g/dose given once per week to once every 12 weeks;
wherein the active vitamin D compound is a compound of formula (III): 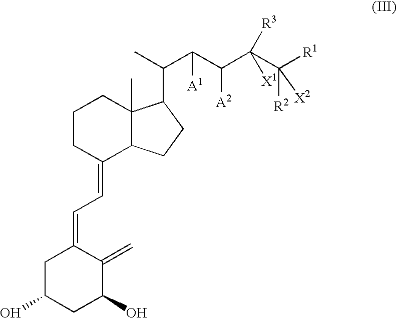
wherein A¹ and A² each are hydrogen or together represent a carbon-carbon bond, thus forming a double bond between C-22 and C-23; R¹ and R² are identical or different and are hydrogen, lower alkyl, lower fluoroalkyl, O-lower alkyl, lower alkenyl, lower fluoroalkenyl, O-lower alkenyl, O-lower acyl, O-aromatic acyl, lower cycloalkyl with the proviso that R¹ and R² cannot both be an alkenyl group, or taken together with the carbon to which they are bonded, form a C₃-C₈ cyclocarbon ring; R³ is lower alkyl, lower alkenyl, lower fluoroalkyl, lower fluoroalkenyl, O-lower alkyl, O-lower alkenyl, O-loweracyl, O-aromatic acyl or lower cycloalkyl; X¹ is hydrogen or hydroxyl, or, taken with R³, constitutes a bond when R³ is an alkenyl group, and X² is hydrogen or hydroxyl, or, taken with R¹ or R², constitutes a double bond.

2. The method of claim 1, wherein the malignant cells are associated with cancers of the breast, colon, prostate, lung, pancreas, endometrium, liver, squamous cell carcinoma, myeloid leukemia, melanoma, retinoblastoma, sarcomas of the soft tissues or bone.

3. The method of claim 1 wherein the active vitamin D is 1α-hydroxyvitamin D₂ or 1α,24-dihydroxyvitamin D₂.

4. The method of claim 3 wherein the active vitamin D is 1α-hydroxyvitamin D₂.

5. The method of claim 3 wherein the active vitamin D is 1α,24-dihydroxyvitamin D₂.

6. The method of claim 1 wherein the active vitamin D is 1α-hydroxyvitamin D₄; 1α,25-dihydroxyvitamin D₂; 1α,24,25-trihydroxyvitamin D₂ , 1α,25-dihydroxyvitamin D₄; 1α,24,25-trihydroxyvitamin D₄; 24-hydroxyvitamin D₂; or 24-hydroxyvitamin D₄.

7. The method of claim 1 wherein the active vitamin D is administered to a human cancer patient, the amount of the active vitamin D effective to inhibit the hyperproliferation of the neoplastic cells.

8. The method of claim 1 wherein the active vitamin D compound is administered parenterally or orally in combination with a pharmaceutically acceptable carrier.

9. The method of claim 8 wherein the active vitamin D compound is administered parenterally.

10. The method of claim 9 wherein the active amount of vitamin D compound is administered intravenously.

11. The method of claim 10 wherein the active vitamin D compound is administered orally.
